(19) United States
(12) Patent Application Publication
(10) Pub. No.: US 2017/0367808 A1
(43) Pub. Date: Dec. 28, 2017

(54) SYSTEM AND METHOD FOR EMBOLIC PROTECTION

(71) Applicant: JAVELIN MEDICAL LTD., Yokneam (IL)

(72) Inventors: Guy SHINAR, Ramat Gan (IL); Avraham NETA, Gilon (IL); Dan YAIR, Kefar Kish (IL); Eyal KAUFMAN, Yokneam (IL); Ofer YODFAT, Modi'in (IL); Sagit BRODER, Gedera (IL)

(21) Appl. No.: 15/542,067

(22) PCT Filed: Jan. 6, 2016
(86) PCT No.: PCT/IL16/50016

§ 371 Date: Jul. 6, 2017

Related U.S. Application Data
(60) Provisional application No. 62/100,369, filed on Jan. 6, 2015.
(60) Provisional application No. 62/216,181, filed on Sep. 9, 2015.

(51) Int. Cl. A61F 2/01

(52) U.S. Cl. CPC A61F 2/01; A61F 2230/0091; A61F 2230/0071; A61F 2002/011; A61F 2220/0008; A61F 2002/016; A61F 2230/0008

(57) ABSTRACT

Embodiments of the present disclosure provide a removable embolic protection device for deployment at a body vessel, as well as systems and methods for implanting the device within a vessel of a patient.

### CROSS REFERENCE TO RELATED APPLICATIONS

This application claims priority to U.S. Provisional Patent Application No. 62/100,369, filed Jan. 6, 2015, and entitled “System and Method for Embolic Protection,” and U.S. Provisional Patent Application No. 62/216,181, filed Sep. 9, 2015, and entitled, “System and Method for Embolic Protection.” The present application incorporates herein by reference the disclosures of each of the above-referenced applications in their entireties.

### FIELD OF THE DISCLOSURE

The field of the disclosure is embolic protection devices.

### SUMMARY OF THE DISCLOSURE

Some embodiments of the present disclosure provide an embolic protection device for deployment at a body vessel. The device may comprise a filament made from a super-elastic alloy. The filament may assume axially extended, free, and axially compressed states.

In the axially extended state the filament may have a substantially linear shape configured to fit within the lumen of a thin needle. In the free state the filament may have a shape comprising a helix portion and a linear segment that is approximately collinear with the helix portion's axis.

In the axially compressed state, the helix portion may be shortened compared to the free state by application of compression force along the helix portion's axis. In the axially compressed state the helix portion may be configured for positioning within the vessel and the linear segment may be configured to breach or traverse the vessel wall; the helix portion may trace the shape of an approximate spherical shell configured to snugly fit within the vessel lumen; and the inter-winding distance may be approximately uniform throughout the entire length of the filament. The helix portion may also trace a shape that is not spherical. For example, one or more turn or winding of the helix portion may approximately trace an oblong shape, such as an oval or an ellipse. One or more of the windings may trace a shape obtained by intersecting two cylindrical shells, one of which may have a circular cross section. The other cylindrical shell may have an oblong cross section. For example, the oblong cross section may be elliptical or approximately oval. At least one anchor comprising one or more protrusions configured to engage tissue may be disposed at the proximal end (that is, the end disposed towards the operator) of the linear segment. A pull-wire may be disposed proximally to the anchor. The pull wire may be configured to extend out of the patient's skin. The filament shape in the free and axially compressed states may comprise a distal linear segment that includes the distal end of the filament (that is, the filament end disposed away from the patient's skin). The compressed helix portion may possess among its plurality of helix portion windings a winding having a maximal diameter. The maximal diameter may be less than or equal to the vessel diameter. The length of the helix portion in the free state may exceed the vessel diameter. Contact between the distal end of the filament and the vessel wall may be ensured by virtue of the free state length exceeding the vessel diameter. In this case, the helix portion may be compressed when deployed within the vessel in the axially compressed state, and thus, the distal filament end may be in contact with the vessel wall. The contact between the distal end and the vessel wall induces tissue growth (also known as “neo-intimal growth”) from the vessel wall on the distal end, thereby securing the distal end in place and preventing its mobility even in severe flow conditions. The shape of the helix portion in the free state is, for a given free state helix portion length, a unique shape that yields upon axial compression and shortening of the free helix portion's length to the axially compressed helix portion's length the desired shape of the helix portion in the axially compressed state. An exact mathematical method enabling calculation of the shape of the free state helix portion from the shape of the axially compressed helix portion shape is provided.

The filament may be given its free state shape by heat treating a nitinol wire wrapped around a stainless steel mandrel having a groove corresponding in shape to the free state helix portion shape.

In operation, the device may be implanted within the vessel in the axially compressed state with the helix portion's axis approximately perpendicular to the vessel. Thus, an embolus originating upstream of the vessel whose size exceeds the inter-winding distance of the axially compressed helix portion may be captured and prevented from flowing downstream. The device may be placed in the common carotid artery in order to capture proximately originating emboli and prevent embolic brain stroke.

Some embodiments of the present disclosure provide systems for embolic protection in a patient. The systems may comprise the embolic protection device described above and a delivery device including a needle, a push tube, and a stabilizing tube. The proximal ends of the push and the stabilizing tubes may be rigidly connected (here “proximal” means closer to the operator and “distal” means away from the operator). The needle may be configured to slidably receive within its lumen the filament (in the extended state) and the push tube. The push tube may be configured to slidably receive the pull wire within its lumen. The stabilizing tube may be configured to slidably receive within the distal end of its lumen the proximal end of the needle. The needle may be configured with a sharp tip capable of puncturing skin and the vessel wall.

Some embodiments of the present disclosure provide systems as described above further comprising an automatic insertion means including a rack rigidly connected to the stabilizing tube, an electronics module, a power source, a motor, a gear, and a man machine interface. The gear may be mechanically coupled to the rack for the purpose of translating rotary motion of the motor into linear motion of the filament, whereby the filament may be automatically exteriorized from the needle. The electronics module may direct power from the power source to activate the motor upon command. The systems may comprise a disposable sterile module including the filament and the needle, and a reusable module including the motor, gear, and power source.

Some embodiments of the present disclosure provide a method of embolic protection in a patient comprising using a system as described above to implant at the vessel of a patient an embolic protection device as described above. The device may be implanted such that the helix portion's axis is approximately perpendicular to the direction of the vessel and at least a portion of the pull wire protrudes out of the skin. Tissue from the vessel wall may grow in the vicinity of the filament's contact points or lengths with the vessel wall, thereby further securing the device in place. In particular, tissue may grow around the distal end of the filament, thereby preventing mobility of the distal end. The device may be removed by pulling it out of the patient's body using the pull wire.

Some embodiments of the present disclosure can be combined with one and/or another of the disclosures and teachings of that found in PCT publication nos. WO2013/179137, WO2014/102767, WO2014/111911, and U.S. Provisional Application 62/100,369, all of which are incorporated herein by reference, disclose embolic protection devices comprising a filament, to create yet other embodiments.

### ADVANTAGES OF SOME OF THE EMBODIMENTS OF THE DISCLOSURE

At least some of the embodiments according to the present disclosure have important advantages:
The helix portion of the device according to some embodiments is configured to include an axially compressed state in which everywhere along the filament the inter-winding distance is approximately uniform, thereby optimizing embolic protection.
In some embodiments, in an axially compressed state, the helix portion of the device traces a predetermined shape configured to fit securely within the vessel lumen, and bring about anchoring of the device both by mechanical contact with the vessel wall and growth of tissue in the vicinity of contact points between the filament and the vessel wall. Device mobility and subsequent tilting, flailing, and fatigue fractures that may result are thereby prevented.

### BRIEF DESCRIPTION OF THE DRAWINGS

Embodiments of the disclosure may be better understood with reference to the accompanying drawings and subsequently provided detailed description:

FIGS. 1A-C depict an embolic protection device according to some embodiments of the present disclosure, which may assume (A) an axially extended state, (B) a free state, and (C) an axially compressed state.

FIG. 2A shows an axially compressed helix shape according to some embodiments of the present disclosure.

FIG. 2B shows a free helix shape form which the axially compressed helix shape of FIG. 2A may be obtainable by axial compression according to some embodiments of the present disclosure.

FIG. 2C shows a projection of an axially compressed helix tracing the shape of a spherical shell and having an approximately uniform inter-winding distance throughout the filament length according to some embodiments of the present disclosure.

FIG. 2D shows a projection of a free helix a free helix from which the axially compressed helix of FIG. 2C may be obtainable by axial compression according to some embodiments of the present disclosure.

FIG. 2E shows the inter-winding distance as a function of the winding number in the axially compressed helix of FIGS. 2A and 2C according to some embodiments of the present disclosure.

FIG. 3A shows a filament of an embolic protection device according to some embodiments of the present disclosure configured in the free state.

FIG. 3B shows the finite elements simulation of the shape of the filament of FIG. 3A after the application of axial force compressing its length to be equal with its diameter according to some embodiments of the present disclosure.

FIGS. 4A and 4B respectively show an anchor according to some embodiments of the present disclosure when constrained in a needle and when free of constraining force according to some embodiments of the present disclosure.

FIGS. 5A-I depict an embolic protection device and embolic protection system according to some embodiments of the present disclosure, an embolic protection method, and optionally, a method of embolic protection device removal.

FIGS. 6A-D depict, respectively, isometric, top, side, and front views of the compressed state of an embolic protection device according to some embodiments of the present disclosure, which may possess one or more windings that approximately trace an oblong shape.

FIG. 7A depicts the axially extended state of an embolic protection device according to some embodiments of the present disclosure, which possesses a proximal-most winding configured to contact the “roof” of a blood vessel lumen in at least one point.

FIGS. 7B-E depict, respectively, isometric, side, front, and top views of the compressed (deployed) state of an embolic protection device according to some embodiments of the present disclosure, which possesses a proximal-most winding configured to contact the “roof” of a blood vessel lumen in at least one point.

FIGS. 7F-I depict, respectively, isometric, side, front, and top views of the free state of an embolic protection device according to some embodiments of the present disclosure, which possesses a proximal-most winding configured to contact the “roof” of a blood vessel lumen in at least one point.

### DETAILED DESCRIPTION OF SOME OF THE EMBODIMENTS

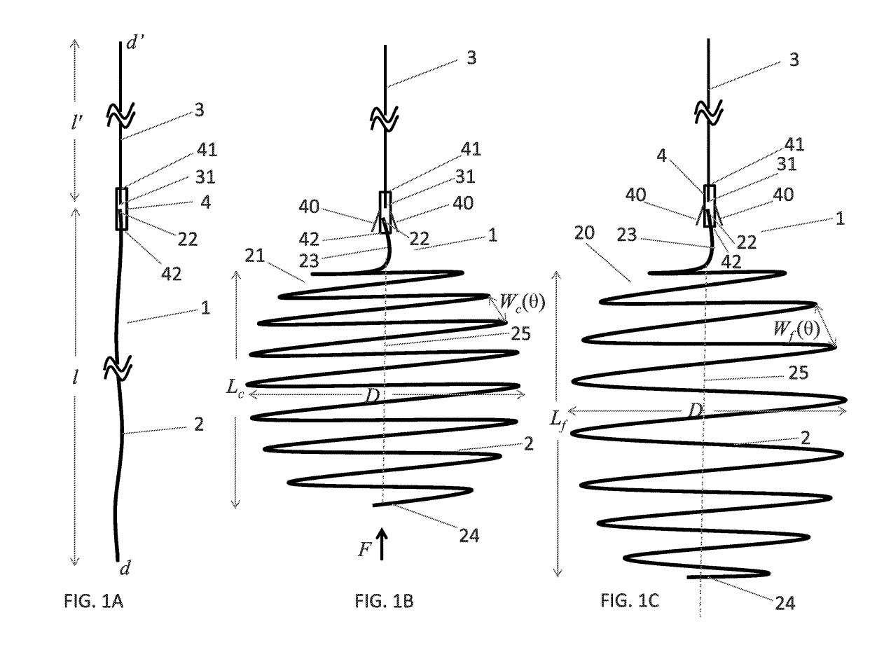

Reference is now made to FIGS. 1A-C, which depict an embolic protection device according to some embodiments of the present disclosure. FIG. 1A depicts an axially extended state of device 1, in which the device, or at least a portion thereof, may be configured to fit within the lumen of a needle. FIG. 1B depicts an axially compressed state of device 1, which may occur in response to a compression force F. FIG. 1C depicts a free state of device 1, which is a state that device 1 may assume in the absence of any forces.

Embolic protection device 1 comprises a filament 2, a pull wire 3 (optional), and one or more anchor 4 (also optional). Filament 2 may be made from a super-elastic alloy such as nitinol. The surface of the filament may be mechanically polished, chemically polished, or electro-polished. Furthermore, the surface of the filament may be passivated in acid. The length of the filament, designated 1 in FIG. 1A, may be, for example, between 1 and 60 cm. The thickness of the filament, designated d in FIG. 1A, may be, for example, in the range of 0.05 and 0.5 mm. Filament 2 may have a circular cross section. Whenever the cross section of the filament is circular the thickness d of the filament is identical with the diameter of the circular cross section.

Filament 2 may assume a substantially linear shape in the axially extended state of FIG. 1A. The substantially linear shape may be induced, for example, by inserting at least a portion of device 1 including filament 2 into the lumen of a thin needle.

Filament 2 may assume, in the axially compressed state of FIG. 1B, a shape comprising a proximal, substantially linear segment 23 and a helix portion 21. Optionally, the shape may also comprise a distal linear segment 24. In some embodiments, when compared to a curved segment, such a linear distal segment facilitates less friction upon exteriorization of device 1 from the lumen of a thin needle.)

The proximal, substantially linear segment may be collinear with axis 25 of helix portion 21 (note that axis 25 is geometrical, not physical). Helix portion 21 may comprise one or more turns or windings. The one or more windings may have different diameters. The length of helix portion 21, denoted L_{c} in FIG. 1B, may be greater than the diameter of the largest winding of helix portion 21, denoted D in FIG. 1B. L_{c} may be equal to D. Helix portion 21 may trace the shape of an approximately spherical shell having a diameter D, in which case L_{c} is approximately equal to D. Helix portion 21 may also trace a non-spherical shape. For example, at least some of the windings of the helix portion may approximately trace an oblong shape, such as an ellipse or an oval. If, for example, a winding approximately traces the shape of an ellipse then the length of the major axis may be roughly equal to the D, and the length of the minor axis may be less than or equal to D. A helix portion in which at least one of the windings approximately traces the shape of a circle, and in which one or more additional windings trace an oblong shape is possible. Helix portion 21 may trace a shape that does not possess rotational symmetry around the helix axis.

Any point along the helix portion may be designated by its winding number θ, which is a coordinate corresponding to the cumulative angular position (in radians) of each point of the helix portion. At the initial point of the helix portion θ=0, at the first winding θ=2π, and at the terminal point of the helix portion θ=2πN, where N, which is not necessarily a whole number, is the number of windings in helix portion 21. The compressed inter-winding distance at point θ, denoted W_{c}(θ), is defined as the axially compressed state distance between point θ and point θ+2π, which is the point exactly one helix portion winding away from θ in the direction of the terminal helix portion point. W_{c}(θ) may be configured to be approximately the same at every point along helix portion 21. In fact in the compressed state W_{c}(θ) may be within +/−15% of a given constant value. W_{c}(θ) may be within +/−10% of a given constant value, or even within +/−5% of a given constant value.

Filament 2 may assume, in the free state of FIG. 1E, a shape comprising the proximal linear segment 23 and a helix portion 20. Optionally, the shape may also comprise the distal linear segment 24. The proximal linear segment may be collinear with axis 25 of helix portion 20, which coincides with the axis of helix 21. Helix portion 20 may comprise the same number of windings as helix portion 21. The one or more windings of helix portion 20 may have different diameters. The length of helix portion 20, designated L_{f} in FIG. 1C, may be greater than the diameter D of the largest winding of helix portion 20, which may be approximately the same as the diameter of the largest winding of helix portion 21. L_{f} may be greater than L_{c}.

The free inter-winding distance W_{f} (θ) at point θ is defined as the free state distance between point θ and the point θ+2π, which is the point exactly one helix portion winding away from θ in the direction of the terminal helix portion point. W_{f}(θ) may vary with the winding number θ. W_{f}(θ) may be greater near the center (the equator) of helix portion 20 than near the poles of helix portion 20. Helix portion 20 may be configured to trace the shape of a body of revolution such that upon transition from the free to the axially compressed state, (1) helix portion 20 transforms into helix portion 21, and/or (2) free (and generally variable) inter-winding distance W_{f}(θ) transforms to axially compressed (and approximately uniform) inter-winding distance W_{c}(θ). Helix portion 20 may also be configured to trace a shape that is not a body of revolution having an axis of revolution collinear with the helix axis.

The ratio of the free length L_{f} and the diameter D may be in the range of 1.0 and 1.5. More specifically, L_{f}/D may be in the range of 1.1 and 1.3.

Distal segment 24 may be approximately perpendicular to helix portion axis 25, both in the free and in the axially compressed states.

In the free state, the radius of curvature at any point along filament 2 may be greater than a certain threshold such that in transition from the free state of FIG. 1C to the axially extended state of FIG. 1A no plastic deformation occurs at any point along filament 2 and the super-elasticity of filament 2 may be maintained. The radius of curvature may exceed the thickness d divided by twice the critical strain of the material from which the filament is made.

The exact shape of helix portion 20 (FIG. 1C) may be obtained from the desired shape of helix portion 21 of the axially compressed state using the precise mathematical model detailed below. Note that once helix portion 21 provides the exact shape of helix portion 20, which is uniquely determined for a given ratio L_{f}/L_{c}.

Example Model

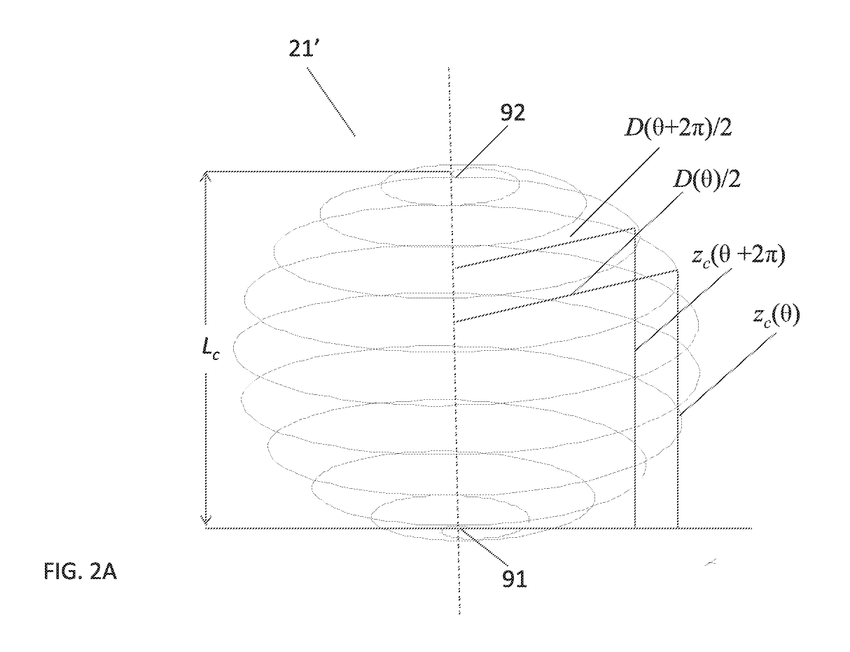

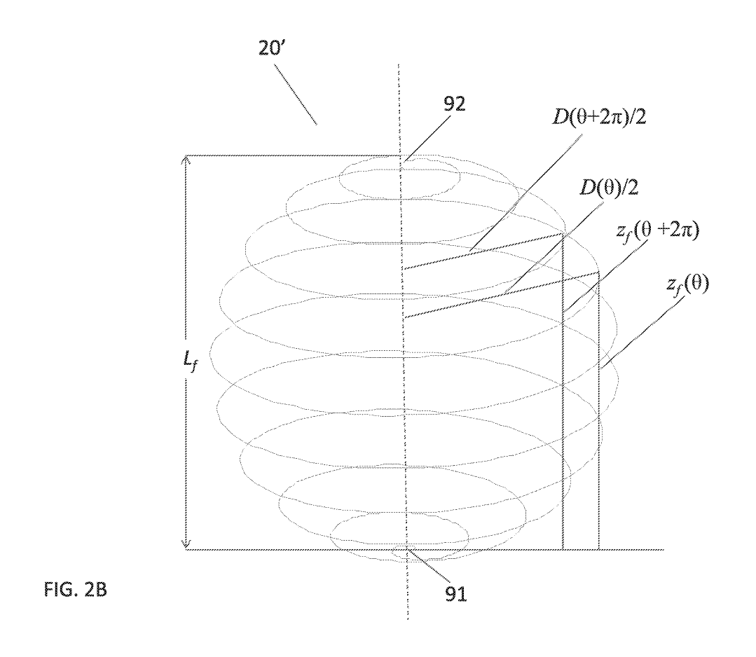

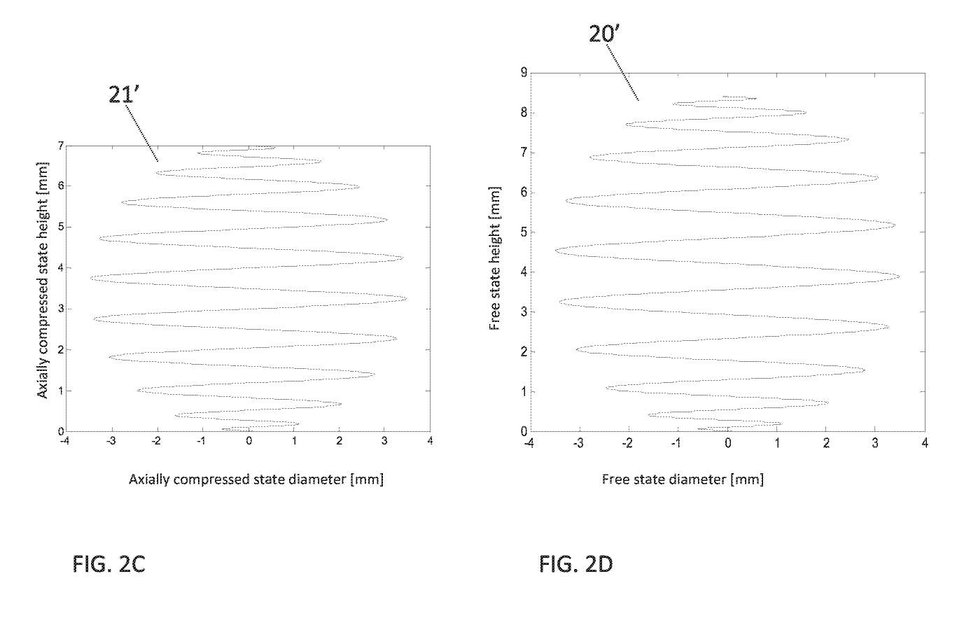

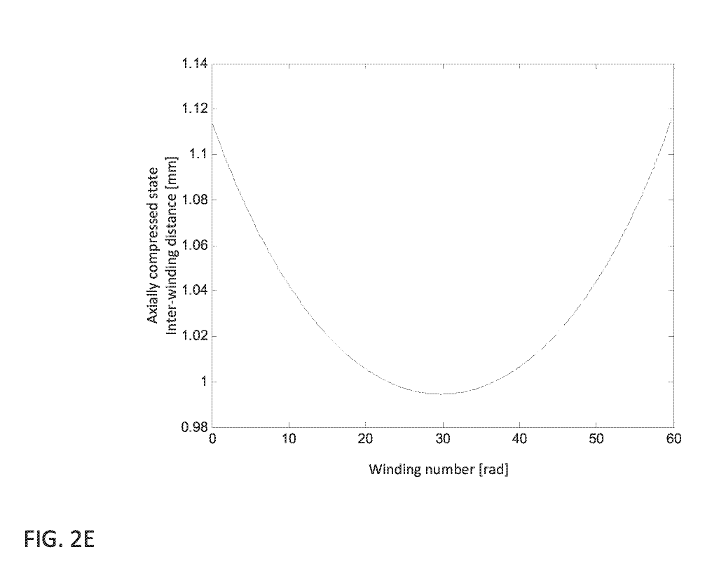

Reference is now made to FIGS. 2A and 2B, which respectively depict an axially-compressed helix shape 21′ and a free helix shape 20′ according to some embodiments of the present disclosure. A goal is to obtain the free state helix shape 20′ from a predetermined axially compressed helix shape 21′ such that under a suitable axial compression force helix 20′ is transformed to helix 21′. Helix portion 20 may then be obtained from helix 20′ by truncating polar portions of helix 20′ for which the radius of curvature at every point is less than the threshold equal to the thickness of filament 2 divided by twice the critical strain of the material from which the filament is made. Appended instead of the discarded polar portions are linear segment 23 and a transition region thereto and, optionally, distal segment 24 and a transition region thereto. Each point along helix 21′ is denoted by its winding number θ. (If the helix has N windings then the winding number of its first end 91 is 0, and the winding number of its second end 92 is 2πN; note that N does not have to be a whole number.)

We denote by z_{c}(θ), θ∈[0,2πN], the height of point θ of helix 21′ and we designate

L_{c}=z_{c}(2πN). (1)

We denote by z_{f}(θ), θ∈[2πN], the height of point θ in the free state helix 20′, and we designate

L_{f}=z_{f}(2πN). (2)

We define the deflection of point θ upon compression of helix 20′ into helix 21′ by

δ(θ=z_{f}(θ)−z_{c}(θ), θ∈[0,2πN]. (3)

### Assumptions:

transition from helix 20′ to helix 21′occurs under constant compression force throughout the entire filament and that the filament thickness may be the same throughout the filament length;
the material properties of the filament are the same throughout the filament's length. We denote by D(θ), θ∈[0,2πN] the diameter of point θ of helix 21′, defined as twice the distance of point θ from the helix axis;
the diameter of point θ in helix 20′ is also equal to D(θ).)

From the theory of elastic springs we have

$$\frac{d δ ( θ )}{d θ} = {AD ( θ )}^{3} + 3 {AD ( θ )}^{2} \frac{dD ( θ )}{d θ} , θ ∈ ( 0 , 2 N ) , \qquad ( 4 )$$

where A is a constant. The constant A can be computed from (1)-(4) as follows:

$$\begin{matrix}{L}_{f} - {L}_{c} = δ ( 2 π N ) = {\int }_{0}^{2 π N} \frac{d δ ( θ )}{d θ} d θ = A {\int }_{0}^{2 π N} ( {D ( θ )}^{3} + 3 {D ( θ )}^{2} \frac{dD ( θ )}{d θ} ) d θ . \\ Thus , & \\ A = \frac{{L}_{f} - {L}_{c}}{{\int }_{0}^{2 π N} ( {D ( θ )}^{3} + 3 {D ( θ )}^{2} \frac{dD ( θ )}{d θ} ) d θ} . & ( 5 )\end{matrix}$$

The free state height z_{f}(θ) of each point θ∈[0,2πN] is obtained from (3):

$${z}_{f} ( θ ) = {z}_{c} ( θ ) + {\int }_{0}^{θ} \frac{d δ ( {θ}^{′} )}{d {θ}^{′}} d {θ}^{′} , \qquad ( 6 )$$

which can be calculated exactly by using (4) and (5) in (6). This provides the exact shape of the free state helix 20′, which is given in Cartesian coordinates by the curve

$$( \frac{D ( θ )}{2} cos ( θ ) , \frac{D ( θ )}{2} sin ( θ ) , {z}_{f} ( θ ) ) , θ ∈ [ 0 , 2 N ] .$$

The inter-winding distance function of helix 21′ is obtained from the Pythagorean theorem:

$${W}_{c} ( θ ) = \sqrt{\frac{{[ D ( θ + 2 π ) - D ( θ ) ]}^{2}}{4} + {[ {z}_{c} ( θ + 2 π ) - {z}_{c} ( θ ) ]}^{2}} , [ θ ∈ 0 , 2 ( N - 1 ) ] . \qquad ( 7 )$$

Similarly, the inter-winding distance function of helix 20′ is

$${W}_{f} ( θ ) = \sqrt{\frac{{[ D ( θ + 2 π ) - D ( θ ) ]}^{2}}{4} + {[ {z}_{f} ( θ + 2 π ) - {z}_{f} ( θ ) ]}^{2}} , [ θ ∈ 0 , 2 ( N - 1 ) ] . \qquad ( 8 )$$

### EXAMPLE

Axially compressed spherical helix shape with approximately uniform inter-winding distance. In such a case helix 20′ may be assumed to trace a spherical shell having length L_{c} equal to diameter D. The pitch function P(θ) which approximates the vertical distance between consecutive windings as a function of θ is chosen such that it is zero at the poles and approximately achieves a value P at the equator. We denote by T the maximal winding number of helix 20′. The following parabolic function is used to prescribe P(θ):

$$P ( θ ) = \frac{4 P}{{T}^{2}} θ ( T - θ ) , θ ∈ [ 0 , 2 N ] . \qquad ( 9 )$$

Note that

$$P ( 0 ) = P ( T ) = 0 , \\ and$$

$$P ( \frac{T}{2} ) = P .$$

z_{c}(θ) is computed as follows from (9):

$${z}_{c} ( θ ) = {\int }_{0}^{θ} \frac{P ( {θ}^{′} )}{2 π} d {θ}^{′} = \frac{P {θ}^{2}}{ π T} - \frac{2 P {θ}^{3}}{3 π {T}^{2}} , θ ∈ [ 0 , 2 N ] . \qquad ( 10 )$$

From (10) and z_{c}(T)=D we obtain

$$T = \frac{3 π D}{P} . \qquad ( 11 )$$

D(θ) is obtained as follows: each point having height z_{c}(θ) and diameter D(θ) must satisfy the equation of a spherical shell, which, for convenience, is centered at point (0, 0, D/2):

$$\begin{matrix}\frac{{D ( θ )}^{2}}{4} + {( {z}_{c} ( θ ) - \frac{D}{2} )}^{2} = \frac{{D}^{2}}{4} , θ ∈ [ 0 , 2 N ] , \\ D ( θ ) = 2 \sqrt{{z}_{c} ( θ ) D - {{z}_{c} ( θ )}^{2} ,} ∈ [ 0 , 2 N ] .\end{matrix} \qquad ( 12 )$$

from which we have
From (4)-(6), and (10)-(12) we obtain helix 21′, which is given in Cartesian coordinates for any choice of parameters L_{f}, D, and P by the curve

$$( \frac{D ( θ )}{2} cos ( θ ) , \frac{D ( θ )}{2} sin ( θ ) , {z}_{f} ( θ ) )$$

for each θ∈[0,2πN]. The inter winding distances in the axially compressed helix 21′ and free helix 20′ are obtained from (7) and (8).

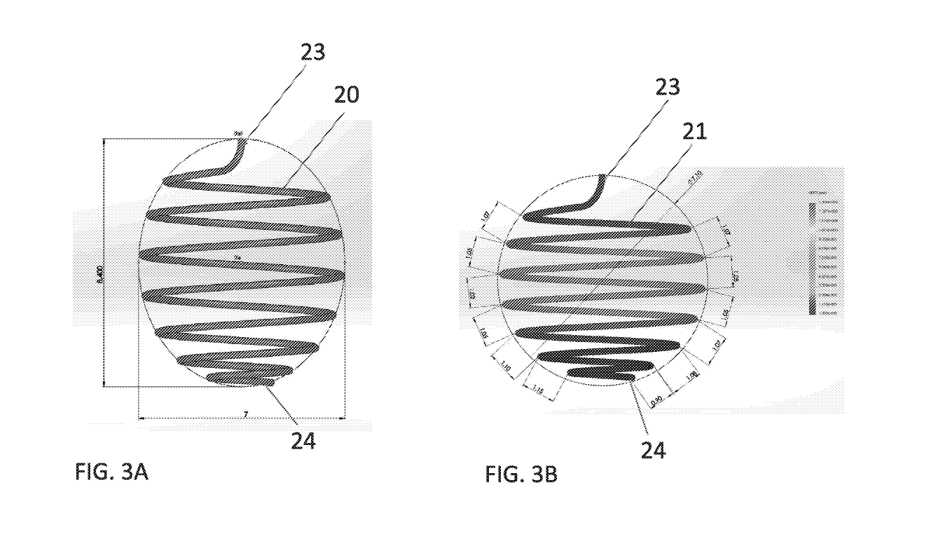

Reference is now made to FIGS. 2C-2E, which present outputs of a model (e.g., see above) for the case in which helix 21′ traces the shape of a spherical shell and its inter-winding distance is approximately uniform. For the purpose of demonstration, the inputs to the model are L_{f}=8.4 mm, D=7 mm, and P=1 mm. FIG. 2C shows that helix 21′ traces the shape of a spherical shell as desired. FIG. 2D shows the unique helix 20′ from which helix 21′ may be obtainable by applying compression force. FIG. 2E shows that the inter-winding distance of helix 21′ is approximately uniform and within the narrow range of about 1.0 and 1.1 mm. Note that the inter-winding distance in the free state (note shown) varies over larger range of 1.1 and 1.3 mm. Reference is now made to FIGS. 3A and 3B. FIG. 3A presents the free state of a filament 2, which may be obtained from helix 20′ by truncating the polar regions resulting in helix portion 20 and appending in their stead a linear segment 23 and a distal linear segment 24. FIG. 3B present the results of a finite elements simulation (SolidWorks) in which filament 2 of FIG. 3A may be axially compressed until the axially compressed length L_{c} is approximately equal to the diameter D. The result of the simulation shows that helix portion 21 approximately traces the shape of a spherical shell, and that the inter-winding distance is approximately uniform. Thus finite element simulations validate the model above as a design tool for the free state of filament 2.

Reference is made again to FIGS. 1A-C. The length of linear segment 23 may be between 0.25 mm and 50 cm. More specifically, the length of linear segment 23 may be between 0.5 and 7 mm.

It is possible to obtain the free state from the compressed state using finite element analysis. This method is especially advantageous whenever the helix portion does not trace the shape of a body of revolution having an axis of revolution identical with the helix axis. First, the compressed state of the device is designed and modeled using finite elements. Then stretching force directed along the helix portion axis is applied using a finite elements simulation, until the desired stretch is obtained. The stretched shape is then identical with the free state shape of the device. Whenever the stretch does not produce plastic deformation, the compressed state may be exactly obtainable from the simulated free (stretched) state by applying compression force directed along the helix portion axis.

Pull wire 3 may be made from a metal. For example, pull wire 3 may be made from a super-elastic alloy such as nitinol or from stainless steel. Pull wire 3 may also be made from a polymer. For example, pull-wire 3 may be made from a natural polymer such as silk, a synthetic polymer such as nylon, or a bio-resorbable polymer such as poly-glycolitic acid. The length of pull wire 3, designated l′ in FIG. 1A, may be, for example, between 0.5 and 50 cm. The thickness of pull wire 3, designated d′ in FIG. 1A, may be in the range of 0.02 and 0.5 mm. Pull wire 3 may have a circular cross section. Whenever the cross section of the pull-wire is circular the thickness d′ of the pull-wire is equal to the diameter of the circular cross section. Pull-wire 3 may be, but does not have to be, integral with filament 2. Thickness d′ of filament 3 may or may not be equal to thickness d of filament 2. d′ may be less than d.

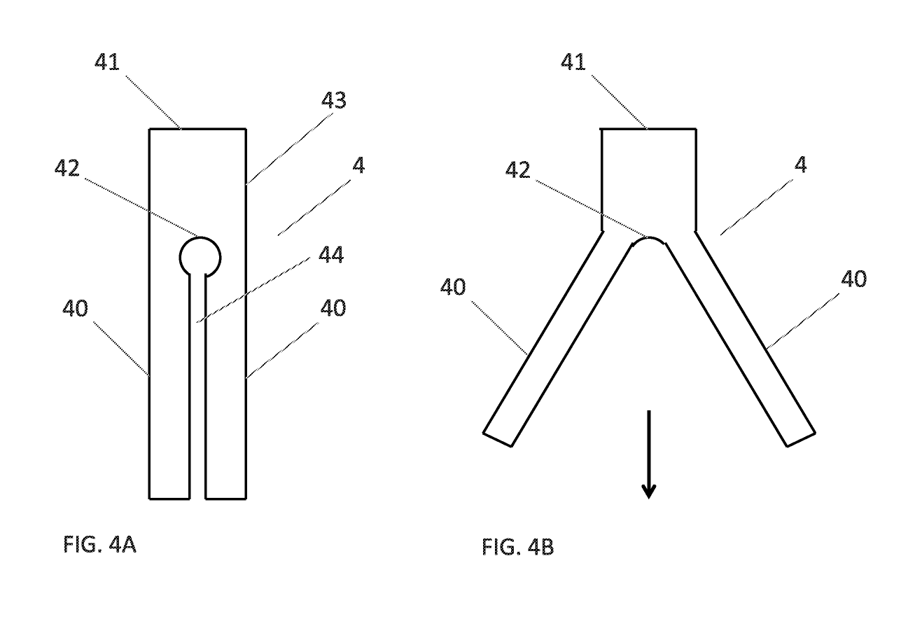

Reference is now made to FIGS. 4A and 4B, which depict respectively an anchor 4 according to some embodiments of the present disclosure. FIG. 4A depicts the shape of the anchor when constrained in the lumen of a needle, corresponding to the axially extended state of filament 2 (FIG. 1A). FIG. 4B depicts the shape of the anchor when unconstrained, as in the free and axially extended states of filament 2B (FIGS. 1C and 1B, respectively).

In the constrained state, anchor 4 has a tubular portion 43 having a proximal end 41 configured to receive the distal end of pull wire 3 and a distal end 42 configured to receive the proximal end of filament 2. Anchor 4 may be attached to each of filament 2 and pull wire 3 by, for example, welding, soldering, brazing or crimping. The anchor may comprise two protrusions 40 separated by slots 44. In the constrained state of FIG. 4A the protrusions lie approximately level or collinear with the walls of tube 43.

Anchor 4 may be made from a super-elastic alloy. Thus, it may be configured to assume when unconstrained the free shape of FIG. 4B, in which protrusions 40 extend outwards and are configured to engage tissue when pulled in the direction of the arrow in FIG. 4B. They are configured to be released from the tissue when pulled in the opposite direction. Anchor 4 may include one or more protrusions and is not limited to having two protrusions as in FIGS. 4A and 4B.

The anchor may be configured to freely rotate around the filament, thereby providing a bearing at the proximal end of the filament. This may be achieved, for example, by welding a ring near the proximal end of the filament, slidably inserting the proximal filament end in the lumen of the anchor, and welding another ring proximally to the anchor. the proximal ring may also serve to connect the filament with the pull wire.

Providing a freely-rotatable anchor is advantageous whenever the helix portion traces a shape that is not a body of revolution having an axis of revolution collinear with the helix axis, which may require that the device assume a particular orientation when implanted in a vessel. For example, whenever the windings of the helix portion are oblong, it may be desired to align their major axis with the vessel axis, which may be greatly aided by the advent of a freely rotatable anchor.

Device 1 may be provided in different sizes. For example, a device 1 intended for placement in the common carotid artery for the purpose of preventing cardio-embolic stroke may have a diameter D in the range of 4-10 mm. Diameter jumps in the range of 0.25 and 0.5 mm are possible.

The thickness of filament 2 may scale linearly with the helix portion diameter D, which results in an approximately uniform helix portion stiffness across the entire range of sizes. This can be seen, for example, by observing that the spring constant of a helix portion scales like

$$\frac{{d}^{4}}{{ND}^{3}} ,$$

where d is the filament thickness, N is the number of windings, and D is the approximate helix portion diameter, in the axially compressed state, also the length. In such embodiments, because the inter-winding distance may be kept constant across the entire range of sizes so that the same minimal embolus size trapped by the device remains the same for the entire size range, the number of windings N also approximately scales like D. Thus, the spring constant scales like

$${( \frac{d}{D} )}^{4} ,$$

and therefore remains uniform whenever d scales like D.

Filament 2 may be manufactured, for example, by heat treating a nitinol wire arranged on a stainless steel mandrel. The mandrel may be configured with a groove shaped as a negative image of the free state of filament 2. Following heat treatment the free state shaped filament may be surface treated by, for example, electro-polishing.

Anchor 4 may be laser cut from a nitinol tube and heat treated on a mandrel configured to give it the free shape depicted in FIG. 4B. The anchor may also be provided with an electro-polished surface finish.

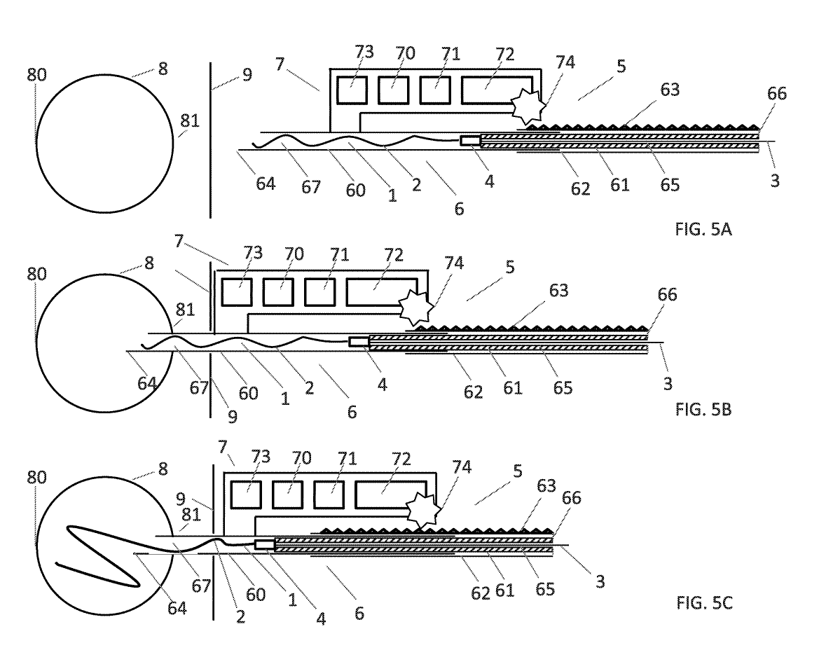

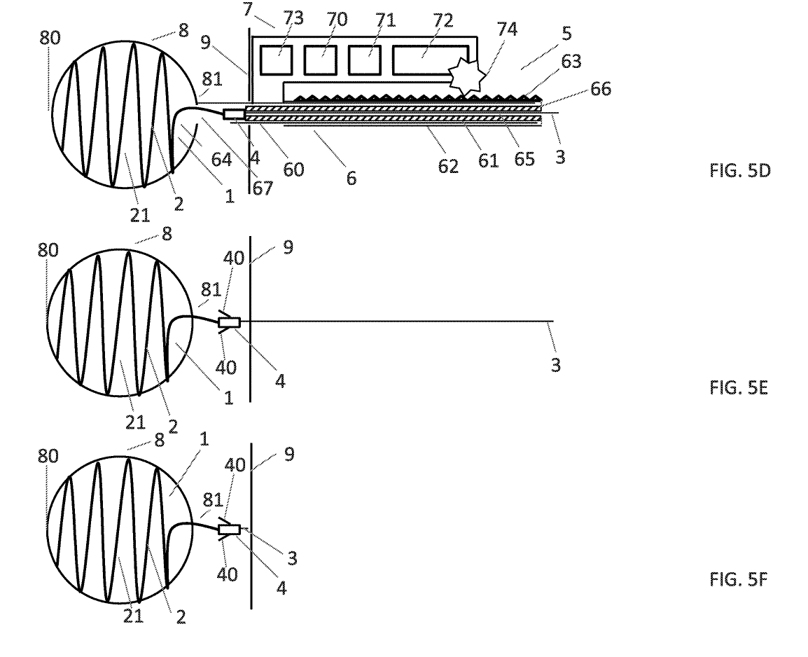

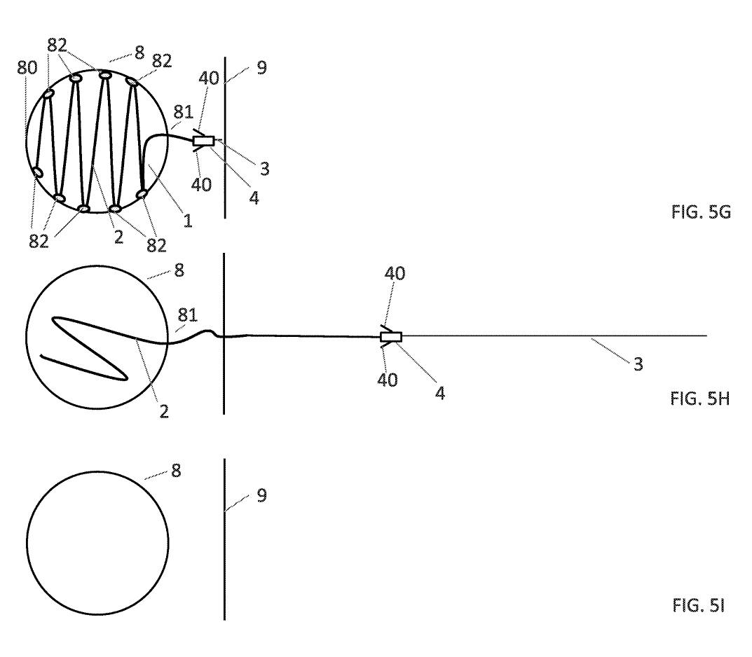

Reference is now made to FIG. 5A, which depicts an embolic protection system 5 according to some embodiments of the present disclosure. System 5 may comprise a disposable module 6 provided sterile, and a reusable module 7 which may or may not be provided sterile. Disposable module 6 and reusable module 7 may be configured to be reversibly engaged by an operator. Reversible engagement may be enabled using, for example, a snap mechanism, a magnet, or pins and holes, and any combinations thereof. Once engaged, the disposable and reusable modules may be rigidly connected.

System 5 may be entirely disposable, with modules 6 and 7 integral with each other, and without the possibility of reversibly engaging and disengaging the modules from each other.

Disposable module 6 may comprise embolic protection device 1, needle 60, push tube 61, stabilizing tube 62, and rack 63. Disposable module 6 may also comprise a reinforcing tube (not shown) having a lumen, wherein at least a portion of needle 60 is within the lumen of the reinforcing tube. Reusable module 7 may comprise a power source 70, electronics module 71, motor 72, and gear 74. Either reusable module 6 or disposable module 7 may comprise a man-machine interface 73. Man machine interface may also be realized as a standalone component (for example a remote controller, wirelessly communicating with a transceiver within the electronic module).

Needle 60 may be made from, for example, metal or plastic. Suitable metals include, for example, stainless steel and nitinol. The needle may have a sharp end 64, configured to penetrate tissue. The needle may possess a lumen 67. The outer diameter of the needle may be between 0.1 and 1 mm. The inner diameter of the needle may vary between 0.2 and 0.9 mm.

Push tube 61 may be made from, for example, metal or plastic. Suitable metals include, for example, stainless steel and nitinol. Push tube 61 may have a lumen 65 extending therethrough.

Stabilizing tube 62 may be made from, for example, metal or plastic. Suitable metals include, for example, stainless steel and nitinol. Stabilizing tube 62 and push tube 61 may be rigidly connected at their proximal ends 66.

Rack 63 may be rigidly connected to stabilizing tube 62. The rack may comprise teeth configured to engage a gear wheel of gear 74.

Needle 60 may be configured to slidably receive within lumen 67 at least a portion of device 1 at its axially extended state. The distal tip of filament 2 may be placed close to tip 64. Anchor 4 may be disposed within lumen 67. At least a portion of pull wire 3 may also be disposed within lumen 67. Needle 60 may also be configured to slidably receive push tube 61 within lumen 67. The distal end of push tube 61 may be placed within lumen 67 proximally to anchor 4. Lumen 65 may be configured to slidably receive at least a portion of pull wire 3. Pull wire 3 may extend proximally to the proximal end 66 of push tube 61. Stabilizing tube 62 may be configured to slidably receive at its distal end the proximal end of needle 60, and, in some embodiments, at least a portion of the reinforcing tube (not shown).

Power source 70 may be a battery. The battery may or may not be rechargeable. Examples of suitable non-rechargeable batteries include batteries based on the following chemistries: zinc-carbon, zinc-chloride, zinc-manganese dioxide, zinc-manganese dioxide/nickel oxyhydroxide, lithium-copper oxide, lithium-iron disulfide, lithium-manganese dioxide, lithium-carbon fluoride, lithium-chromium oxide, mercury oxide, zinc-air, Zamboni pile, silver-oxide, and magnesium. Examples of suitable rechargeable batteries include: nickel-cadmium, lead-acid, nickel-metal hydride, nickel-zinc, silver-oxide, and lithium ion. Whenever the battery is rechargeable, charging leads may be provided in reusable module 7, and a charger may be provided with system 5. An inductive charging mechanism may also be provided.

Electronics module 71 may comprise an integrated circuit, a microprocessor, a controller, and combinations thereof. The microprocessor may include a central processing unit and a memory. Optionally, electronics module 71 may include a receiver, such as a Bluetooth radio.

Motor 72 may be, for example, an electrical motor. Examples of suitable electrical motors may include the following types: shunt, separately excited, series, permanent magnet, induction, synchronous, stepper, brushless DC, hysteresis, reluctance, and universal.

Man-machine interface 73 may comprise an operating button configured to instruct electronics module 71 to cause the exteriorization of device 1 from the needle. The man machine interface may be disposed in disposable module 6 or reusable module 7. Alternatively, man machine interface 73 may be disposed neither on the disposable or the reusable modules: it might be disposed on an ultrasound transducer or as a foot pedal. Optionally, man-machine interface 73 may include a transmitter, such as a Bluetooth radio.

Gear 74 may comprise a gear wheel, configured to translate rotary motion from motor 72 to linear motion of device 1 via rack 63 and push tube 61. Gear 74 may comprise teeth configured to engage corresponding teeth on rack 63. Whenever disposable module 6 and reusable module 7 are engaged, the teeth of the gear wheel and rack 63 may also be engaged.

System 5 may also comprise one or more sensors. For example, system 5 may comprise a motor current sensor, a pH sensor, a pressure sensor, or an impedance sensor, potentially in fluid communication with the needle lumen, and any combination thereof. The system may also comprise a translucent chamber enabling visual inspection of the presence of blood in the needle lumen.

In some embodiments, system 5 may be used by a single operator. Implantation of device 1 may require an imaging modality such as ultrasound, x-ray radiography, x-ray fluoroscopy, computed tomography, magnetic resonance imaging, and any combinations thereof. According to some embodiments, implantation (and potential removal) of the device may proceed as follows:

The operator assesses the target vessel 8 of a patient using an imaging modality. The dimensions of the vessel are measured. If the vessel is, for example, an artery, then the minimal diameter of the artery measured in the course of a blood flow pulse may be recorded.

The operator chooses a system 5 including a device 1 sized as follows: the diameter D is less than the diameter of vessel 8, but no more than 1 mm less than the vessel diameter. The free length L_{f} is greater than the vessel diameter. Such sizing ensures that the device deploys properly (as in FIG. 5D), and that following deployment the device may be in a compressed state wherein the distal end of the device maintains contact with the far vessel wall 80 at all times.

The operator assembles disposable module 6 and reusable module 7 together.

The operator images the implantation site using an imaging modality. Once a clear image of the implantation site is obtained, the operator punctures the skin, the tissue surrounding the implantation site, and the vessel. The vessel puncture may be made such that needle 60 is approximately perpendicular to the vessel. Needle tip 64 is placed in the lumen of vessel 8, as depicted in FIG. 5B.

Once satisfactory needle position is achieved, the operator instructs system 5 via man machine interface 73 to release device 1 from needle 62. Electronics module 71 commands motor 72 to spin gear 74 in the clockwise direction. This causes rack 63, stabilizing tube 62, and push tube 61 to move in the direction of needle tip 64, thereby pushing device 1 out of needle 60, as depicted in FIG. 5C.

Push tube 65 continues to advance towards needle tip 64 until it reaches a pre-determined distal most position. At this point electronics module 71 causes motor 72 to stop. Anchor 4 remains within the lumen of needle 60. At least a portion of proximal linear segment 23 also remains within the lumen of needle 60. The helix portion of device 1 is in the compressed state 21. Helix portion 21 may be deployed within the lumen of vessel 8 such that its axis 25 is approximately collinear with proximal linear segment 23. Device 1 is in an axially compressed state wherein its compressed length L_{c} is approximately equal to the vessel diameter and is less than the free length L_{f}. Contact between the distal end of filament 2 and the far wall 80 of vessel 8 is ensured. The situation is depicted in FIG. 5D.

The operator pulls system 5 away from the patient. The proximal-most winding of helix portion 20 apposes the proximal vessel wall 81 and static friction between needle 60 and anchor 4 is overcome. Pull-wire 3 Slides out of lumen 65 of push tube 61. Once anchor 4 exits the lumen of needle 60 its one or more protrusion 40 protrudes outward and engages the surrounding tissue. The operator continues to pull system 5 backwards until disposable module 6 and reusable module 7 completely disengage from device 1. The situation is as depicted in FIG. 5E: proximal linear segment 23 traverses the wall of vessel 80. Anchor 4 is deployed externally to the vessel and under the skin. Pull wire 3 traverses the patient's skin.

The operator interrogates device 1 using an imaging modality anytime from minutes to weeks after the situation of FIG. 5E is achieved. If the operator finds the result satisfactory, pull wire 3 may be cut at the level of the skin and the skin is lifted such that filament 2, anchor 4, and the remaining portion of pull-wire 3 attached to the anchor or to the filament are all beneath the skin. The implantation procedure is complete. Within one week to several months tissue 82 may grow the vicinity of one or more of the contact points between helix portion 21 and the wall of vessel 8. The tissue growth further secures device 1 to the vessel wall.

If following imaging assessment the operator finds the implantation result unsatisfactory, device 1 may be extracted from the patient's body by pulling on pull wire 3. As device 1 is retracted through the original puncture line through the skin and the vessel wall, at least a portion of filament 2 is straightened. The situation is as in FIG. 5H. Once the device is completely extracted, as in FIG. 51, the operator may attempt a repeat implantation of a device 1.

Device 1 provides embolic protection by filtering emboli originating upstream of the device and preventing them from flowing downstream of the device. Emboli exceeding in size the inter-winding distance in the compressed state are filtered and are prevented from causing damage downstream of the device. For example. Whenever the device is implanted in a common carotid artery embolic protection against cardio-embolic stroke is provided.

Whenever the imaging modality used is ultrasound, system 5 may be operated using one hand and an ultrasound probe may be held in the other hand.

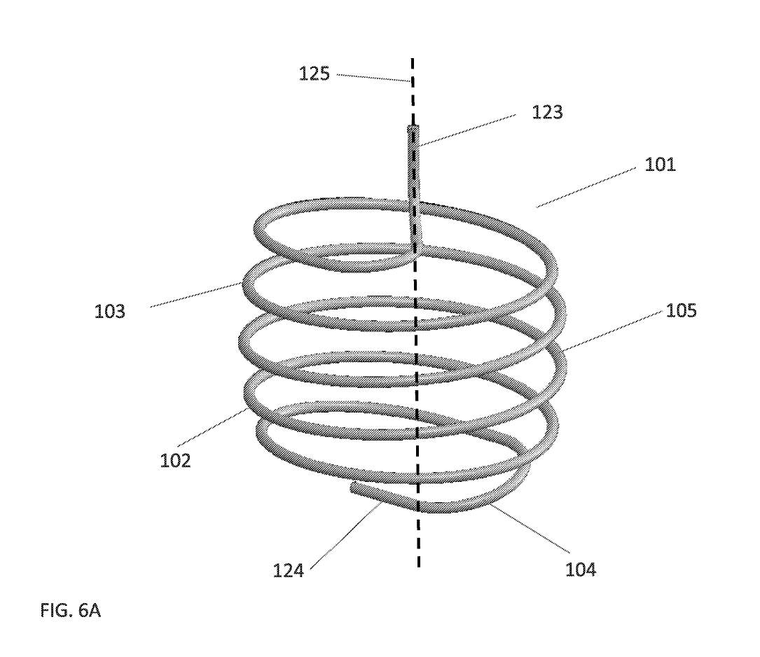

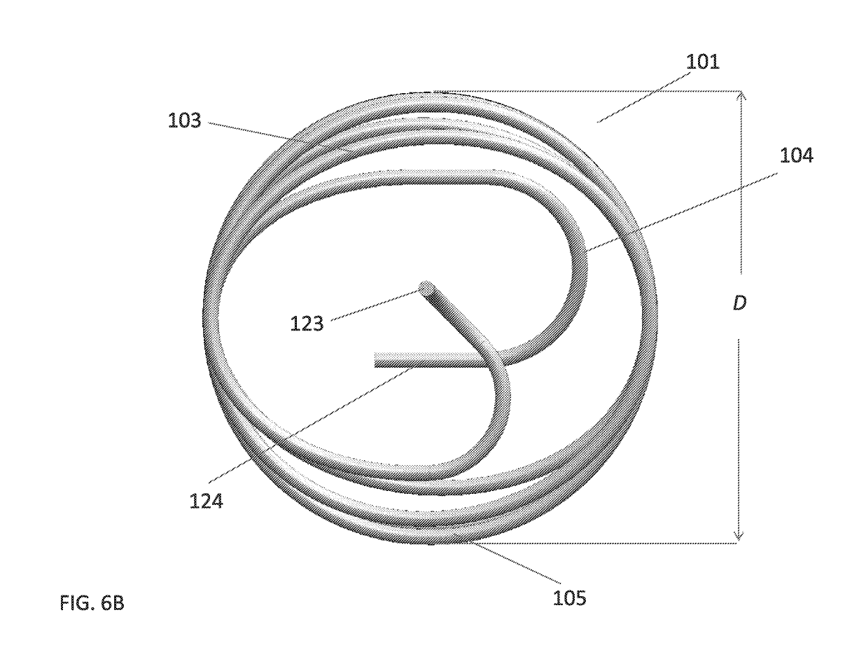

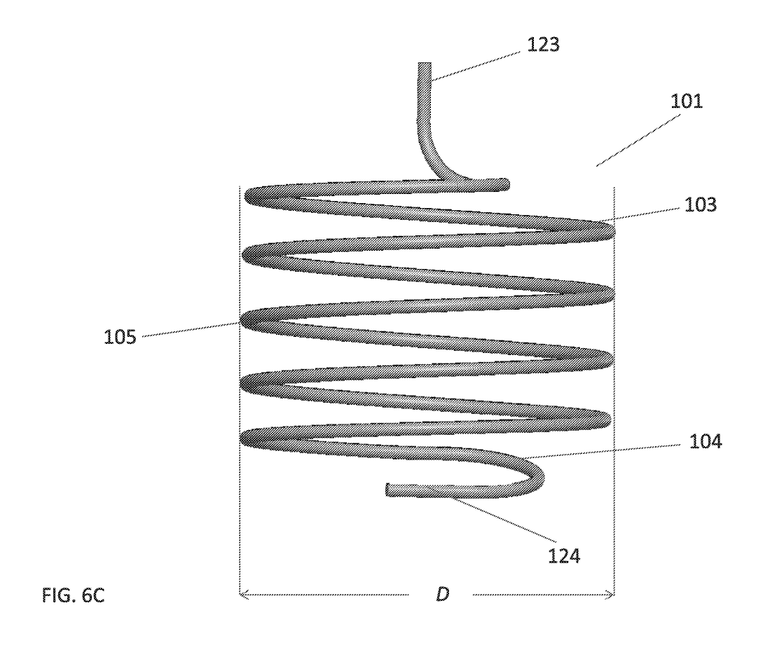

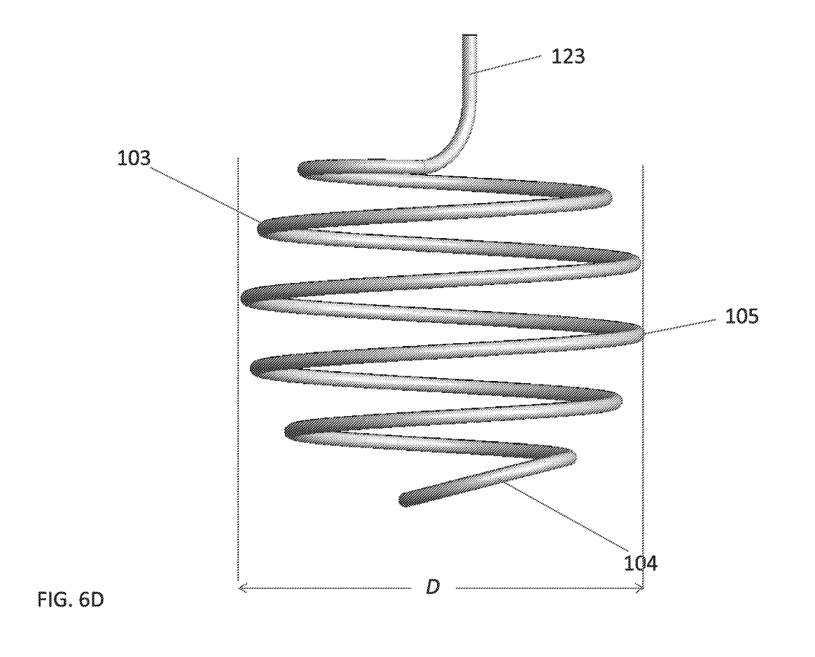

Reference is now made to FIGS. 6A-D, which depict, respectively, isometric, top, side, and front views of the compressed state of an embolic protection device 101 according to some embodiments of the present disclosure. An anchor, which may or may not be freely rotatable, and a pull wire, may essentially be as described above and therefore their detailed description is omitted.

Device 101 is similar to device 1. It comprises a filament that is substantially similar to filament 2. The axially extended state of device 101 is substantially similar to the axially extended state of device 1.

In the compressed state device 101 may comprise a proximal linear segment 123, which is substantially similar to the proximal linear segment 23 of device 1. Device 101 may also comprise a helix portion 121. Helix portion 121 may comprise a plurality of windings, at least one of which may approximately trace an oblong shape. For example, the second winding 103 located distally to segment 123 may approximately trace an oblong shape, such as an ellipse. The distal-most winding 104 may also approximately trace an oblong shape, such as an oval. Equatorial winding 105 may approximately trace the shape of a circle having a diameter D. The major axis of one or more of the oblong windings may have a length approximately equal to D. The minor axis may have a length less than or equal to D.

Distal linear segment 124 may be configured to be approximately perpendicular to helix axis 125 and approximately parallel to the major axis of an oblong shaped winding such as 103.

Device 101 may be configured such that the majority of windings have a major axis length equal to D, and D may be chosen less than or equal to the diameter of a target vessel.

Device 101 may be implanted in substantially similar fashion as device 1 using a substantially similar delivery device. Upon exteriorization from the delivery device into the lumen of a vessel the device will tend to assume a configuration having minimal elastic energy in which the distal linear segment 124 is collinear with the vessel axis. This minimal elastic energy configuration will be realized by the force exerted on the device by the walls of the vessel. A freely rotatable anchor may aid in achieving this configuration.

The free state of device 101 may be obtained from the compressed state of FIG. 6 by “stretching” it along axis 125 using a finite elements simulation.

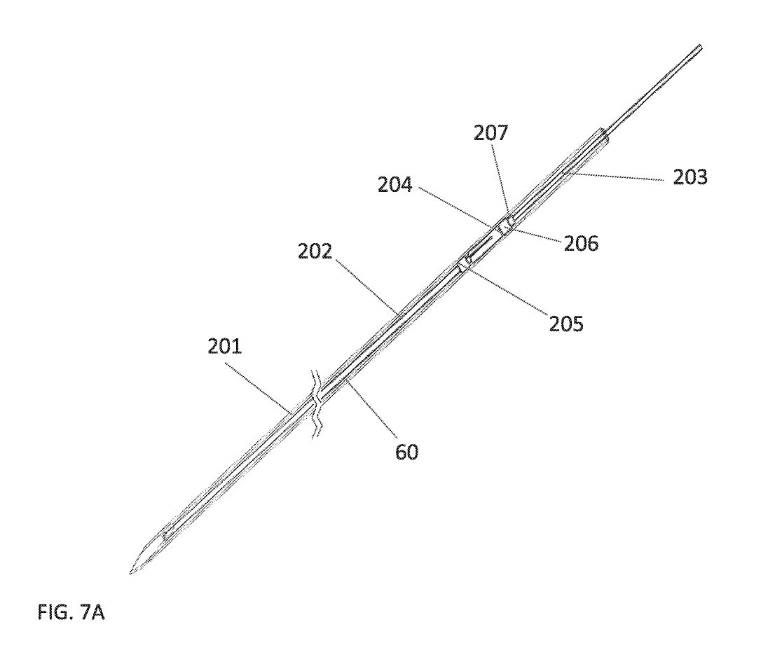

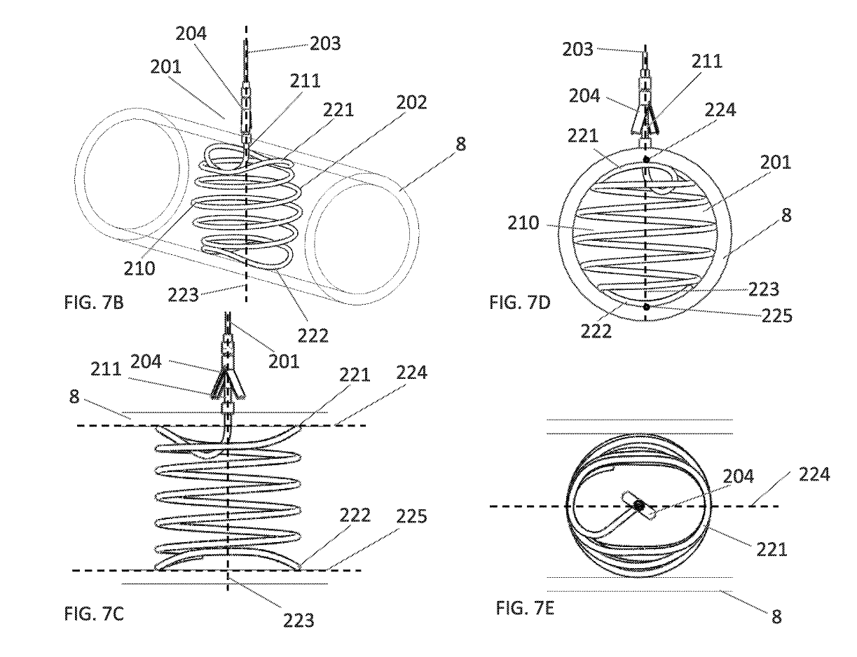

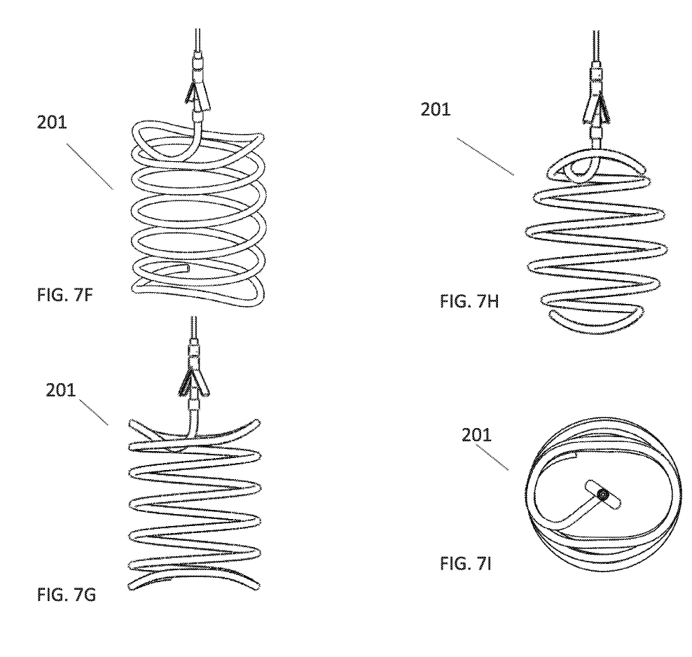

Reference is now made to FIGS. 7A-I, which depict an embolic protection device according to some embodiments of the present disclosure. FIG. 7A depicts the axially extended state of embolic protection device 201 when loaded in a needle 60. Device 201 may comprise a filament 202, a pull wire 203, an anchor 204, distal bushing 205, a proximal bushing 206, and an adaptor bushing 207. FIGS. 7B-E depict, respectively, isometric, side, front, and top views of the compressed state of device 201 when deployed in a blood or body vessel 8. FIGS. 7F-I depict, respectively, isometric, side, front, and top views of the free state of device 201.

Filament 202 may be substantially similar to filament 2, and therefore its detailed description is omitted. The same goes for pull wire 203, which may be substantially similar to pull wire 3, explained above.

Distal bushing 205 may be rigidly connected to filament 202 by any suitable method known in the art, such as, for example, by welding or crimping. The distal brushing 205 may be connected to the distal end of filament 202. Proximal bushing 206 may be connected to, for example, the proximal end of filament 202 by any suitable method known in the art, such as, for example, by welding or crimping. Anchor 204 may be substantially similar to anchor 4, and therefore its detailed description is omitted. Anchor 204 may be positioned between distal and proximal bushings 205 and 206, respectively. Anchor 204 may be freely rotatable around filament 202. Alternatively, in some embodiments, anchor 204 may be rigidly connected to filament 202 by any suitable method known in the art, such as, for example, crimping or welding. In some embodiments where anchor 204 is rigidly connected to filament 202 distal bushing 205 may be unnecessary and may be excluded. In such embodiments anchor 204 may be fixed on filament 202 in any orientation. In particular, the anchor may be fixed perpendicular to the blood vessel axis, parallel to the blood vessel axis, or in any angle in between.

The distal end of pull wire 203 may be connected to proximal bushing 206 by any suitable method known in the art, such as crimping or welding. Optionally, adaptor bushing 207 may be utilized to facilitate the connection between pull wire 203 and proximal bushing 206. Optionally, pull wire 3 may be integral with filament 202.

In the compressed state of device 201, filament 202 may assume a shape comprising a linear segment 211 and a helix portion 210 (see, e.g., FIGS. 7B-E). Linear segment 211 may be configured to breach or traverse the wall of vessel 8. Linear segment 211 may be approximately collinear with axis 223 of helix portion 210. Anchor 204 may be configured to reside externally to the lumen of vessel 8.

Helix portion 210 may comprise a plurality of windings, at least one of which may approximately trace an oblong shape such as an ellipse or an oval. The major axis of one or more of the oblong windings may have a length approximately equal to the diameter of the lumen of vessel 8. The minor axis may have a length less than or equal to the diameter of the lumen of vessel 8.

All or a portion of proximal-most winding 221, all or a portion of distal most winding 222, or all or a portion of each of 221 and 222 may contact or approximately contact the interior of the wall of vessel 8. Proximal-most winding 221, distal most winding 222, or both may approximately trace a shape obtained from the intersection of a cylindrical shell having a circular cross section and a cylindrical shell having an oblong cross section, such as, for example, an ellipse or an oval. The circular cross section may be of the same diameter as the lumen of blood vessel 8.

The terms “proximal-most generator line” or “roof” of the lumen of vessel 8 may be used to describe a line that is parallel to the axis of the vessel and intersects the inner vessel wall at the site where linear segment 211 breaches the inner vessel wall. The terms “distal-most generator line”, or “floor” of the lumen of vessel 8 may be used to describe a line that is parallel to the axis of the vessel and intersects the inner vessel wall at a point diametrically opposed the site where linear segment 211 breaches the inner vessel wall. The terms “roof” and “floor” may also indicate the close vicinity of the aforementioned generator lines.

Proximal-most winding 221 may be configured to contact the most proximal generator line 224 (roof) of the lumen of vessel 8 in at least one point. Distal-most winding 222 may be configured to contact the most distal generator line 225 (floor) of the lumen of vessel 8 in at least one point.

The free state of device 201 (FIGS. 7F-I) may be obtained from the compressed state by “stretching” it along axis 223. For example, when using a finite elements simulation, the stretching factor may be in the range of 1.01 and 2. More specifically, the stretching factor may be in the range of 1.2 and 1.4.

Device 201 may be implanted in substantially similar fashion as device 1 using a substantially similar delivery device. Upon exteriorization from the delivery device into the lumen of a vessel the device will tend to assume a configuration having minimal elastic energy in which proximal-most winding 221, distal-most winding 222 or both are configured to contact the inner wall of vessel 8 throughout the majority of their length (FIG. 7B). In this configuration the major axis of one or more oblong windings is parallel to the axis of vessel 8. This minimal elastic energy configuration will be realized by the force exerted on the device by the walls of the vessel. A freely rotatable anchor may aid in achieving this configuration. However, an anchor rigidly connected to filament 202 may also enable the device to obtain its minimal energy configuration upon exteriorization in the vessel lumen.

Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with features and claims supported by the present disclosure and their equivalents. Moreover, embodiments of the subject disclosure may include methods, systems and devices which may further include any and all elements/features from any other disclosed methods, systems, and devices, including any and all features corresponding to user-experience functionality/systems/methods, including the manufacture and use thereof. In other words, features from one and/or another disclosed embodiment may be interchangeable with features from other disclosed embodiments, which, in turn, correspond to yet other embodiments. One or more features/elements of disclosed embodiments may be removed and still result in patentable subject matter (and thus, resulting in yet more embodiments of the subject disclosure). Furthermore, some embodiments of the present disclosure may be distinguishable from the prior art by specifically lacking one and/or another feature, functionality or structure which is included in the prior art (i.e., claims directed to such embodiments may include “negative limitations”).

1. An embolic protection device for deployment at a human blood vessel, the device comprising a filament having proximal and distal ends, the filament being configured to include an axially extended state and a deployed state wherein:
in the axially extended state, at least a portion of the filament is configured to fit within the lumen of a needle, and
in the deployed state, the filament is configured to form a shape comprising a helix having an axis and a plurality of windings including a proximal-most winding, wherein
the proximal-most winding is configured to contact the roof of the vessel lumen in at least one point.

2. The device of claim 1, wherein the shape further comprises a linear segment proximal to the helix.

3. The device of claim 2, wherein the linear segment is approximately collinear with the axis.

4. The device of claim 2, wherein the linear segment is configured to breach or traverse the vessel wall.

5. The device of claim 1, further comprising an anchor disposed near the proximal end of the filament.

6. The device of claim 5, wherein in the deployed state the anchor is configured to engage tissue external to the blood vessel.

7. The device of claim 5, wherein the anchor comprises one or more protrusions.

8. The device of claim 5, wherein the anchor is made from a slotted tube.

9. The device of claim 5, wherein the anchor is freely-rotatable around the filament.

10. The device of claim 5, wherein the anchor is rigidly fixed to the filament.

11. The device of claim 1, further comprising a pull wire.

12. The device of claim 11, wherein the pull wire is configured to extend out of a patient's skin.

13. The device of claim 11, wherein the pull wire is made from one or more of: a super elastic alloy, a polymer, and a biodegradable polymer.

14. The device of claim 11, wherein the length of the pull wire is between 0.5 and 50 cm.

15. The device of claim 11, wherein the pull wire has a thickness and the thickness of the pull wire is between 0.02 and 0.5 mm.

16. The device of claim 11, wherein the thickness of the pull wire is less than the thickness of the filament.

17-31. (canceled)

32. A system for providing embolic protection in a patient, the system comprising:
the embolic protection device of claim 1;
a needle having proximal and distal ends and a lumen;
a push tube having proximal and distal ends and a lumen; and
a stabilizing tube having proximal and distal ends and a lumen, the stabilizing tube being rigidly connected to the push tube at their respective proximal ends;
wherein
the needle is configured to slidably receive the filament and the push tube within the needle lumen,
the push tube is proximal to the filament within the needle lumen,
the push tube is configured to slidably receive the pull wire within the push tube lumen, and
the stabilizing tube is configured to slidably receive in its distal end the proximal end of the needle.

33. The system of claim 32, further comprising:
a rack rigidly connected to the stabilizing tube;
an electronics module comprising a controller, a microprocessor, an integrated circuit, and any combinations thereof;
a power source;
a motor;
a gear; and
a man machine interface;
wherein
the gear is mechanically coupled to the rack for translating rotary motion of the motor into linear motion of the embolic protection device, whereby the device is automatically exteriorized from the needle; and
the electronics module may direct power from the power source to activate the motor upon command from the man machine interface.

34. The system of claim 33, wherein the device, the needle, the push tube, the stabilizing tube, and the rack are configured as a disposable module, and the electronics module, the power source, the motor, and the gear are configured as a reusable module.

35. The system of claim 34, wherein the disposable module is provided sterile.

36-38. (canceled)
